(19) United States
(12) Patent Application Publication
(10) Pub. No.: US 2024/0207192 A1
(43) Pub. Date: Jun. 27, 2024

(54) PHARMACEUTICAL COMPOSITIONS CONTAINING A PIPERIDINYL-METHYL-PURINE AMINE AND THEIR USE IN TREATING DISEASES AND CONDITIONS

(71) Applicant: K36 THERAPEUTICS, INC., Cambridge, MA (US)

(72) Inventors: Chad Arthur Lewis, Boylston, MA (US); Terrence Joseph Connolly, Concord, MA (US); Anil Bhaskar Menon, Cary, NC (US)

(21) Appl. No.: 18/536,781

(22) Filed: Dec. 12, 2023

Related U.S. Application Data
(60) Provisional application No. 63/431,771, filed on Dec. 12, 2022.

(51) Int. Cl. A61K 9/20; A61K 31/52

(52) U.S. Cl. CPC A61K 9/2059; A61K 9/2013; A61K 9/2054; A61K 9/2095; A61K 31/52

(57) ABSTRACT

The invention provides pharmaceutical compositions comprising a piperidinyl-methyl-purine amine and their use for inhibiting NSD2 and treating a disease or condition, such as cancer.

### CROSS-REFERENCE TO RELATED APPLICATIONS

This application claims the benefit of and priority to U.S. Provisional Patent Application Ser. No. 63/431,771, filed Dec. 12, 2022, the contents of which are hereby incorporated by reference in their entirety.

### FIELD OF THE INVENTION

The invention provides pharmaceutical compositions comprising a piperidinyl-methyl-purine amine and their use for inhibiting NSD2 and treating a disease or condition, such as cancer.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. Current treatment options for these cancers are not effective for all patients and/or can have substantial adverse side effects. New therapies are needed to address this unmet need in cancer therapy.

The nuclear receptor-binding SET domain protein 2 (NSD2), also known as multiple myeloma SET domain (MMSET) or Wolf-Hirschhorn syndrome candidate 1 (WHSC1), is an epigenetic modifier having a role in oncogenesis. Several human cancers are associated with NSD2 overexpression and/or activating point mutations. (Coussens et al., J. Biol. Chem. 293 (2018) 13750-13654.) For example, high expression of NSD2 has been reported in human cancers including bladder, brain, gastrointestinal, lung, liver, ovary, skin, uterus, breast, prostrate and glioblastoma. Additionally, pediatric cancer genomes appear to be particularly likely to contain NSD2 mutations. Finally, upregulation of NSD2 has been linked with aggressive tumor behavior and poor clinical outcomes. Certain compounds that inhibit NSD2 are described in international patent application publication WO 2021/028854. Pharmaceutical compositions of such compounds, having desirable properties for commercial production, would be beneficial to patients suffering from an NSD2-related disease or condition.

The present invention addresses the foregoing needs and provides other related advantages.

### SUMMARY

The invention provides pharmaceutical compositions comprising a piperidinyl-methyl-purine amine and their use for inhibiting NSD2 and treating a disease or condition, such as cancer. In particular, one aspect of the invention provides a pharmaceutical composition, comprising:
(a) at least 10% w/w of a compound of Formula I, wherein Formula I is represented by:

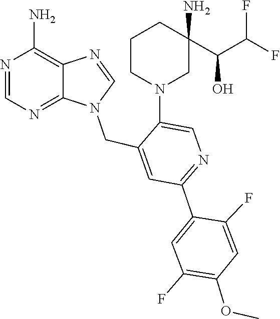 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof;
(b) at least 40% w/w microcrystalline cellulose;
(c) at least 10% w/w pregelatinized starch;
(d) at least 2% w/w croscarmellose or a pharmaceutically acceptable salt thereof; and
(e) at least 0.75% w/w stearic acid or a pharmaceutically acceptable salt thereof.

Further description of additional pharmaceutical compostions are described in the detailed description. For example, another aspect of the invention provides a pharmaceutical composition, comprising:
(a) from 17% w/w to 25% w/w of a compound of Formula I, wherein Formula I is a D-tartaric acid salt of

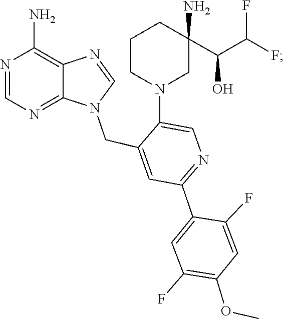 [SMILES: COc1cc(F)c(-c2cc(Cn3cnc4c(N)ncnc43)c(N3CCC[C@](N)([C@H](O)C(F)F)C3)cn2)cc1F]

(b) from 51% to 56% w/w microcrystalline cellulose;
(c) about 20% w/w pregelatinized starch;
(d) about 4% w/w croscarmellose sodium; and
(e) about 1.5% w/w magnesium stearate.

Yet another aspect of the invention provides a pharmaceutical composition, comprising:
(a) at least 10% w/w of a compound of Formula I, wherein Formula I is represented by:

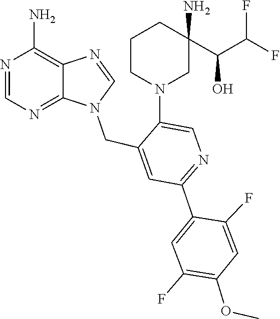 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof; and
(b) at least 60% w/w of a diluent selected from microcrystalline cellulose, pregelatinized starch, or a combination thereof.

Another aspect of the invention provides a tablet for oral administration, wherein the tablet comprises a pharmaceutical composition described herein.

Another aspect of the invention provides a method of preparing a pharmaceutical composition, comprising the steps of:
(i) providing a first mixture comprising:
   (a) at least 10% w/w of a compound of Formula I, wherein Formula I is represented by:

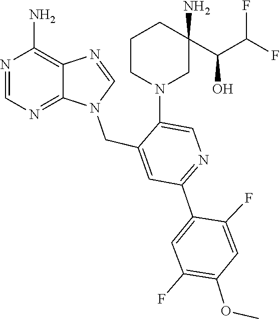 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof;
   (b) at least 40% w/w microcrystalline cellulose;
   (c) at least 10% w/w pregelatinized starch;
   (d) at least 2% w/w croscarmellose or a pharmaceutically acceptable salt thereof; and
   (e) at least 0.75% w/w stearic acid or a pharmaceutically acceptable salt thereof;
(ii) subjecting the first mixture to roller compaction to produce a compacted mixture; and
(iii) subjecting the compacted mixture to compressive force to produce a compressed pharmaceutical composition.

Another aspect of the invention provides a pharmaceutical composition prepared according to the aforementioned method.

Another aspect of the invention provides a method of treating a disease or condition mediated by NSD2 in a subject. The method comprises administering a therapeutically effective amount of a pharmaceutical composition described herein to a subject in need thereof to treat the disease or condition, as further described in the detailed description.

Another aspect of the invention provides a method of inhibiting the activity of nuclear SET domain-containing protein 2 (NSD2). The method comprises contacting a NSD2 with an effective amount of a pharmaceutical composition described herein, as further described in the detailed description.

### DETAILED DESCRIPTION

The invention provides pharmaceutical compositions comprising a piperidinyl-methyl-purine amine and their use for inhibiting NSD2 and treating a disease or condition, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

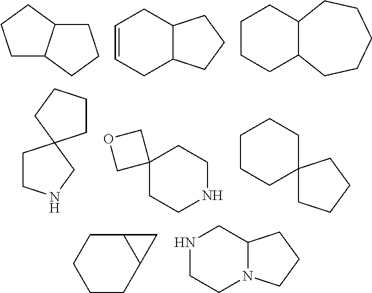

Exemplary bridged bicyclics include:

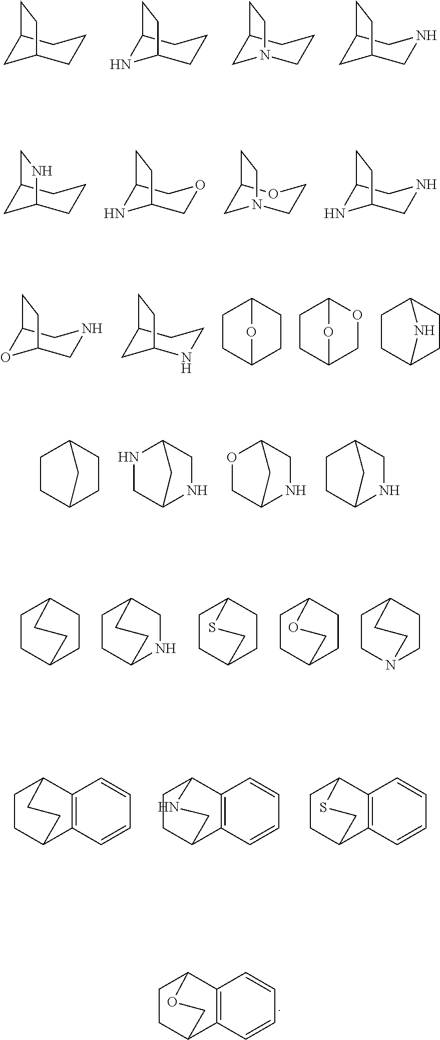

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H pyrrolyl), NH (as in pyrrolidinyl), or NR (as in N substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{·}, -(haloR^{·}), —(CH₂)₀₋₂OH, —(CH₂)₀₋₂OR^{·}, —(CH₂)₀₋₂CH(OR^{·})₂; —O(haloR^{·}), —CN, —N₃, —(CH₂)₀₋₂C(O)R^{·}, —(CH₂)₀₋₂C(O)OH, —(CH₂)₀₋₂C(O)OR^{·}, —(CH₂)₀₋₂SR^{·}, —(CH₂)₀₋₂SH, —(CH₂)₀₋₂NH₂, —(CH₂)₀₋₂NHR^{·}, —(CH₂)₀₋₂NR^{·}₂, —NO₂, —SiR^{·}₃, —OSiR^{·}₃, —C(O)SR^{·}, —(C₁₋₄ straight or branched alkylene)C(O)OR^{·}, or —SSR^{·}.

Each R^{·} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{·} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{·}, -(haloR^{·}), —OH, —OR^{·}, —O(haloR^{·}), —CN, —C(O)OH, —C(O)OR^{·}, —NH₂, —NHR^{·}, —NR^{·}₂, or —NO₂, wherein each R^{·} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{·} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{·}, -(haloR^{·}), —OH, —OR^{·}, —O(haloR^{·}), —CN, —C(O)OH, —C(O)OR^{·}, —NH₂, —NHR^{·}, —NR^{·}₂, or —NO₂, wherein each R^{·} is independently selected from C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur, and wherein each R^{·} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as an atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

Unless specified otherwise, the term “about” refers to within ±10% of the stated value. The invention encompasses embodiments where the value is within ±9%, ±8%, ±7%, ±6%, ±5%, ±4%, ±3%, ±2%, or ±1% of the stated value.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The terms “alkenyl” and “alkynyl” are art-recognized and refer to unsaturated aliphatic groups analogous in length and possible substitution to the alkyls described above, but that contain at least one double or triple bond respectively.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like. The term “hydroxyalkoxyl” refers to an alkoxyl group that is substituted with at least one hydroxyl. Exemplary hydroxyalkoxyl groups include —OCH₂CH₂OH, —OCH₂C(H)(OH)CH₂CH₂OH, and the like. The term “alkoxylene” refers to a bivalent alkoxyl group.

The term “oxo” is art-recognized and refers to a “=O” substituent. For example, a cyclopentane susbsituted with an oxo group is cyclopentanone.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and, most preferably, includes humans.

As used herein, the term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

As used herein, the term “effective amount” refers to the amount of a compound sufficient to effect beneficial or desired results (e.g., a therapeutic, ameliorative, inhibitory, or preventative result). An effective amount can be administered in one or more administrations, applications, or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Pharmaceutical Compositions

The invention provides pharmaceutical compositions comprising a piperidinyl-methyl-purine amine. The pharmaceutical compositions may be used in the therapeutic methods described herein. Exemplary pharmaceutical compositions are described in the following sections, along with exemplary procedures for making the pharmaceutical compositions.

### Part A—First Pharmaceutical Composition

One aspect of the invention provides a pharmaceutical composition, comprising:
(a) at least 10% w/w of a compound of Formula I, wherein Formula I is represented by:

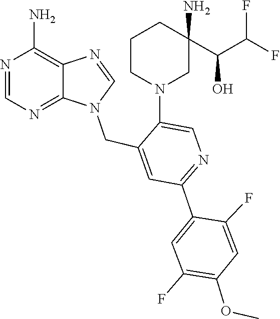 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof;
(b) at least 40% w/w microcrystalline cellulose;
(c) at least 10% w/w pregelatinized starch;
(d) at least 2% w/w croscarmellose or a pharmaceutically acceptable salt thereof; and
(e) at least 0.75% w/w stearic acid or a pharmaceutically acceptable salt thereof.

The First pharmaceutical composition may be further described according to additional features, such as the amounts and identities of the constituents of the pharmaceutical composition. A more thorough description of such features is provided below. The invention embraces all permutations and combinations of these features.

For example, in certain embodiments, the pharmaceutical composition comprises at least 50% w/w microcrystalline cellulose. In certain embodiments, the pharmaceutical composition comprises from 45% to 65% w/w microcrystalline cellulose. In certain embodiments, the pharmaceutical composition comprises from 50% to 56% w/w microcrystalline cellulose. In certain embodiments, the pharmaceutical composition comprises from 52% to 54% w/w microcrystalline cellulose. In certain embodiments, the pharmaceutical composition comprises about 53% w/w microcrystalline cellulose. In certain embodiments, the pharmaceutical composition comprises 53% w/w microcrystalline cellulose.

In certain embodiments, the pharmaceutical composition comprises at least 15% w/w pregelatinized starch. In certain embodiments, the pharmaceutical composition comprises at least 20% w/w pregelatinized starch. In certain embodiments, the pharmaceutical composition comprises from 15% to 25% w/w pregelatinized starch. In certain embodiments, the pharmaceutical composition comprises from 18% to 22% w/w pregelatinized starch. In certain embodiments, the pharmaceutical composition comprises about 20% w/w pregelatinized starch. In certain embodiments, the pharmaceutical composition comprises 20% w/w pregelatinized starch.

In certain embodiments, the pharmaceutical composition comprises at least 4% w/w croscarmellose or a pharmaceutically acceptable salt thereof. In certain embodiments, the pharmaceutical composition comprises from 3% w/w to 5% w/w croscarmellose or a pharmaceutically acceptable salt thereof. In certain embodiments, the pharmaceutical composition comprises about 4% w/w croscarmellose or a pharmaceutically acceptable salt thereof. In certain embodiments, the pharmaceutical composition comprises 4% w/w croscarmellose or a pharmaceutically acceptable salt thereof.

In certain embodiments, the croscarmellose or a pharmaceutically acceptable salt thereof is croscarmellose sodium.

In certain embodiments, the pharmaceutical composition comprises at least 1% w/w of stearic acid or a pharmaceutically acceptable salt thereof. In certain embodiments, the pharmaceutical composition comprises at least 1.5% w/w of stearic acid or a pharmaceutically acceptable salt thereof. In certain embodiments, the pharmaceutical composition comprises from 1% to 2% w/w of stearic acid or a pharmaceutically acceptable salt thereof. In certain embodiments, the pharmaceutical composition comprises about 1.5% w/w of stearic acid or a pharmaceutically acceptable salt thereof. In certain embodiments, the pharmaceutical composition comprises 1.5% w/w of stearic acid or a pharmaceutically acceptable salt thereof.

In certain embodiments, the stearic acid or a pharmaceutically acceptable salt thereof is an alkaline earth metal salt of stearic acid. In certain embodiments, the stearic acid or a pharmaceutically acceptable salt thereof is magnesium stearate.

In certain embodiments, the pharmaceutical composition comprises at least 15% w/w of a compound of Formula I. In certain embodiments, the pharmaceutical composition comprises at least 20% w/w of a compound of Formula I. In certain embodiments, the pharmaceutical composition comprises from 10% to 30% w/w of a compound of Formula I. In certain embodiments, the pharmaceutical composition comprises from 15% to 25% w/w of a compound of Formula I. In certain embodiments, the pharmaceutical composition comprises from 18% to 24% w/w of a compound of Formula I. In certain embodiments, the pharmaceutical composition comprises from 20% to 22% w/w of a compound of Formula I. In certain embodiments, the pharmaceutical composition comprises about 21% w/w of a compound of Formula I. In certain embodiments, the pharmaceutical composition comprises 21% w/w of a compound of Formula I.

In certain embodiments, the compound of Formula I is a tartaric acid salt of

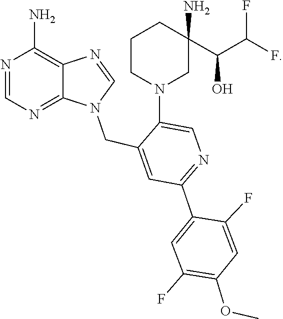 [SMILES: COc1cc(F)c(-c2cc(Cn3cnc4c(N)ncnc43)c(N3CCC[C@](N)([C@H](O)C(F)F)C3)cn2)cc1F]

In certain embodiments, the compound of Formula I is a D-tartaric acid salt of

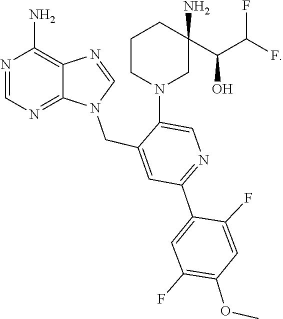 [SMILES: COc1cc(F)c(-c2cc(Cn3cnc4c(N)ncnc43)c(N3CCC[C@](N)([C@H](O)C(F)F)C3)cn2)cc1F]

In certain embodiments, the compound of Formula I is a L-tartaric acid salt of

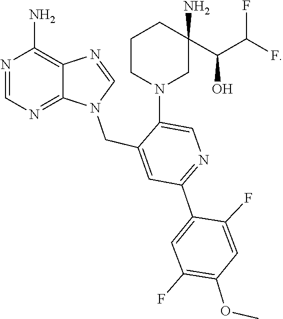 [SMILES: COc1cc(F)c(-c2cc(Cn3cnc4c(N)ncnc43)c(N3CCC[C@](N)([C@H](O)C(F)F)C3)cn2)cc1F]

In certain embodiments, the compound of Formula I is a fumaric acid salt of

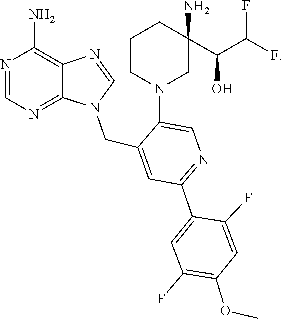 [SMILES: COc1cc(F)c(-c2cc(Cn3cnc4c(N)ncnc43)c(N3CCC[C@](N)([C@H](O)C(F)F)C3)cn2)cc1F]

### Part B—Second Pharmaceutical Composition

Another aspect of the invention provides a pharmaceutical composition, comprising:
(a) from 17% w/w to 25% w/w of a compound of Formula I, wherein Formula I is a D-tartaric acid salt of

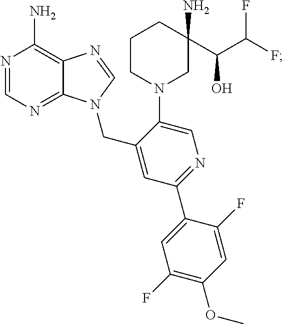 [SMILES: COc1cc(F)c(-c2cc(Cn3cnc4c(N)ncnc43)c(N3CCC[C@](N)([C@H](O)C(F)F)C3)cn2)cc1F]

(b) from 51% to 56% w/w microcrystalline cellulose;
(c) about 20% w/w pregelatinized starch;
(d) about 4% w/w croscarmellose sodium; and
(e) about 1.5% w/w magnesium stearate.

The Second pharmaceutical composition may be further described according to additional features, such as the amounts and identities of the constituents of the pharmaceutical composition. A more thorough description of such features is provided below. The invention embraces all permutations and combinations of these features.

For example, in certain embodiments, the pharmaceutical composition comprises from 19% w/w to 23% w/w of the compound of Formula I. In certain embodiments, the pharmaceutical composition comprises from 20% w/w to 22% w/w of the compound of Formula I. In certain embodiments, the pharmaceutical composition comprises about 21% w/w of the compound of Formula I. In certain embodiments, the pharmaceutical composition comprises 21% w/w of the compound of Formula I.

In certain embodiments, the pharmaceutical composition comprises from 52% to 55% w/w microcrystalline cellulose. In certain embodiments, the pharmaceutical composition comprises from 53% to 54% w/w microcrystalline cellulose. In certain embodiments, the pharmaceutical composition comprises 53% w/w microcrystalline cellulose. In certain embodiments, the pharmaceutical composition comprises 53% w/w microcrystalline cellulose.

### Part C—Third Pharmaceutical Composition

Another aspect of the invention provides a pharmaceutical composition, comprising:
(a) at least 10% w/w of a compound of Formula I, wherein Formula I is represented by:

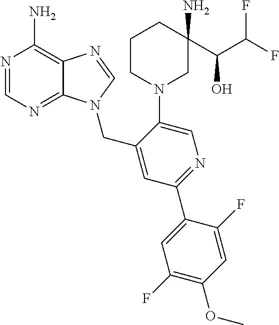 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof; and
(b) at least 60% w/w of a diluent selected from microcrystalline cellulose, pregelatinized starch, or a combination thereof.

The Third pharmaceutical composition may be further described according to additional features, such as the amounts and identities of the constituents of the pharmaceutical composition. A more thorough description of such features is provided below. The invention embraces all permutations and combinations of these features.

For example, in certain embodiments, the pharmaceutical composition further comprises a lubricant. In certain embodiments, the lubricant is stearic acid or a pharmaceutically acceptable salt thereof. In certain embodiments, the lubricant is magnesium stearate.

In certain embodiments, the pharmaceutical composition further comprises a disintegrant. In certain embodiments, the disintegrant is an alkali metal starch glycolate, crospovidone, croscarmellose, or a pharmaceutically acceptable salt thereof, or a combination thereof. In certain embodiments, the disintegrant is an alkali metal starch glycolate. In certain embodiments, the disintegrant is croscarmellose or a pharmaceutically acceptable salt thereof. In certain embodiments, the disintegrant is an alkali metal croscarmellose. In certain embodiments, the disintegrant is crospovidone.

In certain embodiments, the pharmaceutical composition further comprises a binder. In certain embodiments, the binder is polyvinylpyrrolidone, hydroxypropyl cellulose, or a combination thereof. In certain embodiments, the binder is polyvinylpyrrolidone. In certain embodiments, the binder is hydroxypropyl cellulose. In certain embodiments, the binder is a combination of polyvinylpyrrolidone and hydroxypropyl cellulose.

In certain embodiments, the pharmaceutical composition further comprises water.

### Part D—Fourth Pharmaceutical Composition

Another aspect of the invention provides a pharmaceutical composition, comprising:
(a) a compound of Formula I, wherein Formula I is represented by:

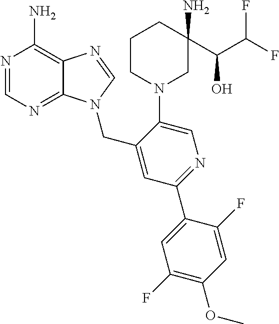 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof; and
(b) a diluent.

### Part E—Additional Features of the First, Second, Third, and Fourth Pharmaceutical Composition

The First, Second, Third, and Fourth pharmaceutical compositions may be further characterized according to additional features, such as bulk density of the pharmaceutical composition, tap density of the pharmaceutical composition, and other features. A more thorough description of such features is provided below. The invention embraces all permutations and combinations of these features.

For example, in certain embodiments, the pharmaceutical composition has a bulk density of at least 0.2 g/cm³. In certain embodiments, the pharmaceutical composition has a bulk density of at least 0.3 g/cm³. In certain embodiments, the pharmaceutical composition has a bulk density of at least 0.4 g/cm³. In certain embodiments, the pharmaceutical composition has a bulk density of at least 0.5 g/cm³. In certain embodiments, the pharmaceutical composition has a bulk density of at least 0.6 g/cm³. In certain embodiments, the pharmaceutical composition has a bulk density in the range of 0.5 to 0.7 g/cm³. In certain embodiments, the pharmaceutical composition has a bulk density in the range of 0.55 to 0.6 g/cm³.

In certain embodiments, the pharmaceutical composition has a tap density of at least 0.2 g/cm³. In certain embodiments, the pharmaceutical composition has a tap density of at least 0.3 g/cm³. In certain embodiments, the pharmaceutical composition has a tap density of at least 0.4 g/cm³. In certain embodiments, the pharmaceutical composition has a tap density of at least 0.5 g/cm³. In certain embodiments, the pharmaceutical composition has a tap density of at least 0.6 g/cm³. In certain embodiments, the pharmaceutical composition has a tap density of at least 0.7 g/cm³. In certain embodiments, the pharmaceutical composition has a tap density of at least 0.8 g/cm³. In certain embodiments, the pharmaceutical composition has a tap density in the range of 0.6 to 0.8 g/cm³. In certain embodiments, the pharmaceutical composition has a tap density in the range of 0.7 to 0.75 g/cm³.

In certain embodiments, the pharmaceutical composition does not contain a phosphate salt. In certain embodiments, the pharmaceutical composition does not contain an alkali or alkaline earth metal phosphate salt. In certain embodiments, the pharmaceutical composition does not contain an alkaline earth metal phosphate salt. In certain embodiments, the pharmaceutical composition does not contain a calcium phosphate salt. In certain embodiments, the pharmaceutical composition does not contain mannitol.

### II. Tablets

Another aspect of the invention provides tablets. Such tablets may contain a pharmaceutical composition described herein and be used in oral administration.

Accordingly, one aspect of the invention provides a tablet for oral administration, comprising a pharmaceutical composition described herein.

In certain embodiments, the tablet has a hardness of at least 6 kP. In certain embodiments, the tablet has a hardness of at least 7 kP. In certain embodiments, the tablet has a hardness of at least 8 kP. In certain embodiments, the tablet has a hardness of in the range of from about 6 to about 8 kP.

In certain embodiments, the tablet contains about 25, about 50, about 75, about 100, about 125, or about 150 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-tartrate. In certain embodiments, the tablet contains about 25 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-tartrate. In certain embodiments, the tablet contains about 50 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-tartrate. In certain embodiments, the tablet contains about 75 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-tartrate. In certain embodiments, the tablet contains about 150 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-tartrate.

In certain embodiments, the tablet contains about 25, about 50, about 75, about 100, about 125, or about 150 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol or a pharmaceutically acceptable salt thereof. In certain embodiments, the tablet contains about 25 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol or a pharmaceutically acceptable salt thereof. In certain embodiments, the tablet contains about 50 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol or a pharmaceutically acceptable salt thereof. In certain embodiments, the tablet contains about 75 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol or a pharmaceutically acceptable salt thereof. In certain embodiments, the tablet contains about 150 mg of (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol or a pharmaceutically acceptable salt thereof.

### III. Methods for Preparing Pharmaceutical Compositions

Another aspect of the invention provides methods for preparing pharmaceutical compositions. For example, one aspect of the invention provides a method of preparing a pharmaceutical composition, comprising the steps of:
(i) providing a first mixture comprising:
   (a) at least 10% w/w of a compound of Formula I, wherein Formula I is represented by:

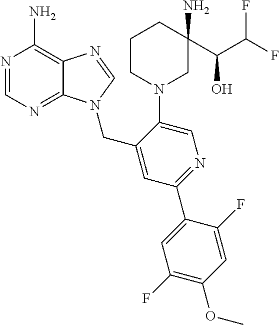 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof;
   (b) at least 40% w/w microcrystalline cellulose;
   (c) at least 10% w/w pregelatinized starch;
   (d) at least 2% w/w croscarmellose or a pharmaceutically acceptable salt thereof, and
   (e) at least 0.75% w/w stearic acid or a pharmaceutically acceptable salt thereof,
(ii) subjecting the first mixture to roller compaction to produce a compacted mixture; and
(iii) subjecting the compacted mixture to compressive force to produce a compressed pharmaceutical composition.

In certain embodiments, the method further comprises the steps of:
(i) providing a starting mixture comprising:
   (a) at least 10% w/w of a compound of Formula I, wherein Formula I is represented by:

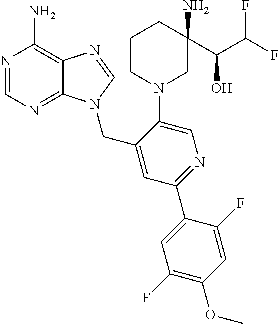 [SMILES: COc1cc(F)c(-c2cc(Cn3cnc4c(N)ncnc43)c(N3CCC[C@](N)([C@H](O)C(F)F)C3)cn2)cc1F]

or a pharmaceutically acceptable salt thereof;
   (b) at least 40% w/w microcrystalline cellulose;
   (c) at least 10% w/w pregelatinized starch;
   (d) at least 2% w/w croscarmellose or a pharmaceutically acceptable salt thereof; and
   (e) at least 0.2% w/w stearic acid or a pharmaceutically acceptable salt thereof;
(ii) applying the starting mixture to a screen to filter out particles having a diameter larger than about 991 microns, to thereby produce a filtered mixture; and
(iii) admixing the filtered mixture with magnesium stearate to form said first mixture.

Another aspect of the the invention provides a pharmaceutical composition prepared according to a method described herein.

### IV. Therapeutic Applications

The pharmaceutical compositions described herein provide therapeutic benefits to subjects suffering from cancer and other diseases or conditions. Accordingly, one aspect of the invention provides a method for treating a disease or condition mediated by nuclear SET domain-containing protein 2 (NSD2). The method comprises administering a therapeutically effective amount of a pharmaceutical composition described herein to a subject in need thereof to treat the disease or condition.

Examples of diseases or conditions that are mediated by NSD2 include but is not limited to breast cancer, cervical cancer, skin cancer (particularly skin squamous cell carcinoma), ovarian cancer, gastric cancer, prostate cancer, pancreatic cancer, lung cancer, hepatocellular carcinoma, head and neck cancer, peripheral nerve sheath tumor, osteosarcoma, multiple myeloma, neuroblastoma, leukemia (particularly acute lymphoblastic leukemia), non-Hodgkin's lymphoma (particularly mantle cell lymphoma), and pulmonary arterial hypertension.

In certain embodiments, said disease or condition mediated by NSD2 is cancer.

In certain embodiments, said disease or condition mediated by NSD2 is selected from a solid tumor, leukemia, myeloma, lymphoma, and hypertension. In certain embodiments, said disease or condition mediated by NSD2 is a solid tumor. In certain embodiments, said disease or condition mediated by NSD2 is selected from leukemia, myeloma, and lymphoma. In certain embodiments, said disease or condition mediated by NSD2 is leukemia. In certain embodiments, said disease or condition mediated by NSD2 is myeloma. In certain embodiments, said disease or condition mediated by NSD2 is lymphoma. In certain embodiments, said disease or condition mediated by NSD2 is hypertension.

In certain embodiments, said disease or condition mediated by NSD2 is breast cancer, cervical cancer, skin cancer, ovarian cancer, gastric cancer, prostate cancer, pancreatic cancer, lung cancer, hepatocellular carcinoma, head and neck cancer, peripheral nerve sheath tumor, osteosarcoma, multiple myeloma, neuroblastoma, leukemia, non-Hodgkin's lymphoma, or pulmonary arterial hypertension. In certain embodiments, said disease or condition mediated by NSD2 is breast cancer. In certain embodiments, said disease or condition mediated by NSD2 is cervical cancer. In certain embodiments, said disease or condition mediated by NSD2 is ovarian cancer. In certain embodiments, said disease or condition mediated by NSD2 is gastric cancer. In certain embodiments, said disease or condition mediated by NSD2 is prostate cancer. In certain embodiments, said disease or condition mediated by NSD2 is pancreatic cancer. In certain embodiments, said disease or condition mediated by NSD2 is hepatocellular carcinoma. In certain embodiments, said disease or condition mediated by NSD2 is head and neck cancer. In certain embodiments, said disease or condition mediated by NSD2 is a peripheral nerve sheath tumor. In certain embodiments, said disease or condition mediated by NSD2 is osteosarcoma. In certain embodiments, said disease or condition mediated by NSD2 is multiple myeloma. In certain embodiments, said disease or condition mediated by NSD2 is neuroblastoma. In certain embodiments, said disease or condition mediated by NSD2 is pulmonary arterial hypertension.

In certain embodiments, said disease or condition mediated by NSD2 is acute lymphoblastic leukaemia, skin squamous cell carcinoma, or mantle cell lymphoma. In certain embodiments, said disease or condition mediated by NSD2 is acute lymphoblastic leukaemia. In certain embodiments, said disease or condition mediated by NSD2 is skin squamous cell carcinoma. In certain embodiments, said disease or condition mediated by NSD2 is mantle cell lymphoma.

In certain embodiments, said disease or condition mediated by NSD2 is lung cancer. In certain embodiments, said disease or condition mediated by NSD2 is small cell or non-small cell lung cancer. In certain embodiments, said disease or condition mediated by NSD2 is small cell lung cancer. In certain embodiments, said disease or condition mediated by NSD2 is non-small cell lung cancer.

In certain embodiments, said disease or condition mediated by NSD2 is leukemia. In certain embodiments, said disease or condition mediated by NSD2 is acute lymphoblastic leukemia (ALL), acute myeloid leukemia (AML), chronic myeloid leukemia (CML), or chronic myelomonocytic leukemia (CMML). In certain embodiments, said disease or condition mediated by NSD2 is AML. In certain embodiments, said disease or condition mediated by NSD2 is CML. In certain embodiments, said disease or condition mediated by NSD2 is CMML.

In certain embodiments, said disease or condition mediated by NSD2 is skin cancer. In certain embodiments, said disease or condition mediated by NSD2 is melanoma, basal cell carcinoma, or squamous cell carcinoma. In certain embodiments, said disease or condition mediated by NSD2 is melanoma. In certain embodiments, said disease or condition mediated by NSD2 is basal cell carcinoma.

In certain embodiments, said disease or condition mediated by NSD2 is lymphoma. In certain embodiments, said disease or condition mediated by NSD2 is Hodgkin's lymphoma or non-Hodgkin's lymphoma. In certain embodiments, said disease or condition mediated by NSD2 is Hodgkin's lymphoma. In certain embodiments, said disease or condition mediated by NSD2 is non-Hodgkin's lymphoma. In certain embodiments, said disease or condition mediated by NSD2 is mantle cell lymphoma or diffuse large B cell lymphoma. In certain embodiments, said disease or condition mediated by NSD2 is diffuse large B cell lymphoma.

In certain embodiments, said disease or condition mediated by NSD2 is myeloma.

In certain embodiments, said disease or condition mediated by NSD2 is thyroid cancer. In certain embodiments, said disease or condition mediated by NSD2 is colon cancer.

In certain embodiments, the cancer overexpresses NSD2. In certain embodiments, the cancer has a mutation in NSD2. In certain embodiments, the cancer has an activating mutation in NSD2. In certain embodiments, the cancer has the t(4; 14)(p16.3; q32.3) translocation in NSD2. In certain embodiments, the cancer has an E1099K mutation in NSD2. In certain embodiments, the cancer has an T1150A mutation in NSD2.

In certain embodiments, the subject is a human. In certain embodiments, the subject is an adult human. In certain embodiments, the subject is a pediatric human. In certain embodiments, the subject is a geriatric human.

Another aspect of the invention provides for the use of a pharmaceutical composition described herein (such as the First, Second, Third, or Fourth pharmaceutical composition, or other pharmaceutical compositions in Section I) in the manufacture of a medicament. In certain embodiments, the medicament is for treating a disease or condition described herein, such as cancer.

Another aspect of the invention provides for the use of a pharmaceutical composition described herein (such as the First, Second, Third, or Fourth pharmaceutical composition, or other pharmaceutical compositions in Section I) for treating a disease or condition, such as a disease or condition described herein (for example, cancer).

Further, pharmaceutical compositions described herein, such as the First, Second, Third, or Fourth pharmaceutical composition, or other pharmaceutical compositions in Section I, inhibit the activity of nuclear SET domain-containing protein 2 (NSD2). Accordingly, another aspect of the invention provides a method of inhibiting the activity of nuclear SET domain-containing protein 2 (NSD2). The method comprises contacting a NSD2 with an effective amount of a pharmaceutical composition described herein, such as the First, Second, Third, or Fourth pharmaceutical composition, or other pharmaceutical compositions in Section I, to inhibit the activity of said NSD2.

Pharmaceutical compositions may be tested for ability to bind to and/or inhibit NSD2 activity according to any of various assays known in the art, including, for example, LC-MS/MS enzymatic assays monitoring SAH production, cellular FRET assays, cellular ELISA assays, methyltransferase enzymatic luminescence assays monitoring SAH production, and radiometric assays using tritium-labeled SAM. Such assays are described in, for example, WO 2021/028854 and Coussens, N. P. et al. J. Biol. Chem. (2018) Vol. 293, No. 35, pp. 13750-13755; the entirety of each of which is hereby incorporated by reference.

### VI. Combination Therapy

Another aspect of the invention provides for combination therapy. Pharmaceutical compositions described herein may be used in combination with additional therapeutic agents to treat diseases or conditions, such as a cancer.

Accordingly, in some embodiments, the present invention provides a method of treating a disclosed disease or condition comprising administering to a patient in need thereof an effective amount of a pharmaceutical compositions disclosed herein and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents.

One or more other therapeutic agents may be administered separately from a pharmaceutical composition of the invention, as part of a multiple dosage regimen. Alternatively, one or more other therapeutic agents may be part of a single dosage form, mixed together with a pharmaceutical composition of this invention in a single composition. If administered as a multiple dosage regime, one or more other therapeutic agent and a compound or composition of the invention may be administered simultaneously, sequentially or within a period of time from one another.

In certain embodiments, the additional therapeutic agent is an anti-cancer agent, anti-allergic agent, anti-nausea agent (or anti-emetic), pain reliever, cytoprotective agent, or a combination thereof. In certain embodiments, the additional therapeutic agent is an anti-cancer agent, an analgesic, an anti-inflammatory agent, or a combination thereof.

In certain embodiments, the additional therapeutic agent is an anti-cancer agent or chemo-therapeutic agent. Examples of anti-cancer agents considered for use in combination therapies of the invention include but are not limited erlotinib, bortezomib, fulvestrant, sunitib, imatinib mesylate, letrozole, finasunate, platins such as oxaliplatin, carboplatin, and cisplatin, finasunate, fluorouracil, rapamycin, leucovorin, lapatinib, lonafamib, sorafenib, gefitinib, camptothecin, topotecan, bryostatin, adezelesin, anthracyclin, carzelesin, bizelesin, dolastatin, auristatins, duocarmycin, eleutherobin, taxols such as paclitaxel or docetaxel, cyclophosphamide, doxorubicin, vincristine, prednisone or prednisolone, other alkylating agents such as mechlorethamine, chlorambucil, and ifosfamide, antimetabolites such as azathioprine or mercaptopurine, other microtubule inhibitors (vinca alkaloids like vincristine, vinblastine, vinorelbine, and vindesine, as well as taxanes), podophyllotoxins (etoposide, teniposide, etoposide phosphate, and epipodophyllotoxins), topoisomerase inhibitors, other cytotoxins such as actinomycin, daunorubicin, valrubicin, idarubicin, edrecolomab, epirubicin, bleomycin, plicamycin, mitomycin, as well as other anticancer antibodies (cetuximab, bevacizumab, ibritumomab, abagovomab, adecatumumab, afutuzumab, alacizumab, alemtuzumab, anatumomab, apolizumab, bavituximab, belimumab, bivatuzumab mertansine, blinatumomab, brentuximab vedotin, cantuzumab mertansine, catumazomab, cetuximab, citatuzumab bogatox, cixutumumab, clivatuzumab tetraxetan, conatumumab, dacetuzumab, daclizumab, detumomab, ecromeximab, edrecolomab, elotuzumab, epratuzumab, ertumaxomab, etaracizumab, farletuzumab, figitumumab, fresolimumab, galiximab, gembatumumab vedotin, gemtuzumab, ibritumomab tiuxetan, inotuzumab ozogamicin, intetumumab, ipilimumab, iratumumab, labetuzumab, lexatumumab, lintuzumab, lucatumumab, lumilisimab, mapatumumab, matuzumab, milatuzumab, mitumomab, nacolomab tafenatox, naptumomab estafenatox, necitumumab, nimotuzumab, ofatumumab, olaratumab, oportuzumab monatox, oregovomab, panitumumab, pemtumomab, pertuzumab, pintumomab, pritumumab, ramucirumab, rilotumumab, robatumumab, rituximab, sibrotuzumab, tacatuzumab tetraxetan, taplitumomab paptox, tenatumomab, ticilimumab, tigatuzumab, tositumomab or ¹³¹I-tositumomab, trastuzumab, tremelimumab, tuocotuzumab celmoleukin, veltuzumab, visilizumab, volocixumab, votumumab, zalutumumab, zanolimumab, IGN-101, MDX-010, ABX-EGR, EMD72000, ior-t1, MDX-220, MRA, H-11 scFv, huJ591, TriGem, TriAb, R3, MT-201, G-250, ACA-125, Onyvax-105, CD:-960, Cea-Vac, BrevaRex AR54, IMC-1C11, GlioMab-H, ING-1, anti-LCG MAbs, MT-103, KSB-303, Therex, KW2871, anti-HMI.24, Anti-PTHrP, 2C4 antibody, SGN-30, TRAIL-RI MAb, Prostate Cancer antibody, H22xKi-r, ABX-Mai, Imuteran, Monopharm-C), and antibody-drug conjugates comprising any of the above agents (especially auristatins MMAE and MMAF, maytansinoids like DM-1, calicheamycins, or various cytotoxins).

In certain embodiments, the additional therapeutic agent is selected from anastrozole (ARIMIDEX®), bicalutamide (CASODEX®), bleomycin sulfate (BLENOXANE®), busulfan (MYLERAN®), busulfan injection (BUSULFEX®), capecitabine (XELODA®), N4-pentoxycarbonyl-5-deoxy-5-fluorocytidine, carboplatin (PARAPLATIN®), carmustine (BiCNU®), chlorambucil (LEUKERAN®), cisplatin (PLATINOL®), cladribine (LEUSTATIN®), cyclophosphamide (CYTOXAN® or NEOSAR®), cytarabine, cytosine arabinoside (CYTOSAR-U®), cytarabine liposome injection (DEPOCYT®), dacarbazine (DTIC-Dome®), dactinomycin (actinomycin D, COSMEGAN®), daunorubicin hydrochloride (CERUBIDINE®), daunorubicin citrate liposome injection (DAUNOXOME®), dexamethasone, docetaxel (TAXOTERE®), doxorubicin hydrochloride (ADRIAMYCIN®, RUBEX®), etoposide (VEPESID®), fludarabine phosphate (FLUDARA®), 5-fluorouracil (ADRUCIL®, EFUDEX®), flutamide (EULEXIN®), tezacitibine, gemcitabine (difluorodeoxycitidine), hydroxyurea (HYDREA®), idarubicin (IDAMYCIN®), ifosfamide (IFEX®), irinotecan (CAMPTOSAR®), L-asparaginase (ELSPAR®), leucovorin calcium, melphalan (ALKERAN®), 6-mercaptopurine (PURINETHOL®), methotrexate (FOLEX®), mitoxantrone (NOVANTRONE®), gemtuzumab ozogamicin (MYLOTARG™), paclitaxel (TAXOL®), nab-paclitaxel (ABRAXANE®), phoenix (Yttrium90/MX-DTPA), pentostatin, polifeprosan 20 with carmustine implant (GLIADEL®), tamoxifen citrate (NOLVADEX®), teniposide (VUMON®), 6-thioguanine, thiotepa, tirapazamine (TIRAZONE®), topotecan hydrochloride for injection (HYCAMPTIN®), vinblastine (VELBAN®), vincristine (ONCOVIN®), and vinorelbine (NAVELBINE®).

In certain embodiments, the additional therapeutic agent is capable of inhibiting BRAF, MEK, CDK4/6, SHP-2, HDAC, EGFR, MET, mTOR, PI3K or AKT, or a combination thereof. In a particular embodiment, the compounds of the present invention are combined with another therapeutic agent selected from vemurafinib, debrafinib, LGX818, trametinib, MEK162, LEE011, PD-0332991, panobinostat, verinostat, romidepsin, cetuximab, gefitinib, erlotinib, lapatinib, panitumumab, vandetanib, INC280, everolimus, simolimus, BMK120, BYL719 or CLR457, or a combination thereof.

In certain embodiments, the additional therapeutic agent is selected based on the disease or condition that is being treated. For example, in the treatment of melanoma, the additional therapeutic agent is selected from aldesleukin (e.g., PROLEUKIN®), dabrafenib (e.g., TAFINLAR®), dacarbazine, recombinant interferon alfa-2b (e.g., INTRON® A), ipilimumab, trametinib (e.g., MEKINIST®), peginterferon alfa-2b (e.g., PEGINTRON®, SYLATRON™), vemurafenib (e.g., ZELBORAF®)), and ipilimumab (e.g., YERVOY®).

For the treatment of ovarian cancer, the additional therapeutic agent is selected from doxorubicin hydrochloride (Adriamycin®), carboplatin (PARAPLATIN®), cyclophosphamide (CYTOXAN®, NEOSAR®), cisplatin (PLATINOL®, PLATINOL-AQ®), doxorubicin hydrochloride liposome (DOXIL®, DOX-SL®, EVACET®, LIPODOX®), gemcitabine hydrochloride (GEMZAR®), topotecan hydrochloride (HYCAMTIN®), and paclitaxel (TAXOL®).

For the treatment of thyroid cancer, the additional therapeutic agent is selected from doxorubicin hydrochloride (Adriamycin®), cabozantinib-S-malate (COMETRIQ®), and vandetanib (CAPRELSA®).

For the treatment of colon cancer, the additional therapeutic agent is selected from fluorouracil (e.g., ADRUCIL®, EFUDEX®, FLUOROPLEX®), bevacizumab (AVASTIN®), irinotecan hydrochloride (CAMPTOSTAR®), capecitabine (XELODA®), cetuximab (ERBITUX®), oxaliplatin (ELOXATIN®), leucovorin calcium (WELLCOVORIN®), regorafenib (STIVARGA®), panitumumab (VECTIBIX®), and ziv-aflibercept (ZALTRAP®).

For the treatment of lung cancer, the additional therapeutic agent is selected from methotrexate, methotrexate LPF (e.g., FOLEX®, FOLEX PFS®, Abitrexate®, MEXATE®, MEXATE-AQ®), paclitaxel (TAXOL®), paclitaxel albumin-stabilized nanoparticle formulation (ABRAXANE®), afatinib dimaleate (GILOTRIF®), pemetrexed disodium (ALIMTA®), bevacizumab (AVASTIN®), carboplatin (PARAPLATIN®), cisplatin (PLATINOL®, PLATINOL-AQ®), crizotinib (XALKORI®), erlotinib hydrochloride (TARCEVA®), gefitinib (IRESSA®), and gemcitabine hydrochloride (GEMZAR®).

For the treatment of pancreatic cancer, the other therapeutic agent may be selected from fluorouracil (ADRUCIL®), EFUDEX®, FLUOROPLEX®), erlotinib hydrochloride (TARCEVA®), gemcitabine hydrochloride (GEMZAR®), and mitomycin or mitomycin C (MITOZYTREX™, MUTAMYCIN®).

For the treatment of cervical cancer, the additional therapeutic agent is selected from bleomycin (BLENOXANE®), cisplatin (PLATINOL®, PLATINOL-AQ®) and topotecan hydrochloride (HYCAMTIN®).

For the treatment of head and neck cancer, the additional therapeutic agent is selected from methotrexate, methotrexate LPF (e.g., FOLEX®, FOLEX PFS®, Abitrexate®, MEXATER, MEXATE-AQ®), fluorouracil (ADRUCIL®, EFUDEX®, FLUOROPLEX®), bleomycin (BLENOXANE®), cetuximab (ERBITUX®), cisplatin (PLATINOL®, PLATINOL-AQ®) and docetaxel (TAXOTERE®).

For the treatment of leukemia, including chronic myelomonocytic leukemia (CMML), the additional therapeutic agent is selected from bosutinib (BOSULIF®), cyclophosphamide (CYTOXAN®, NEOSAR®), cytarabine (CYTOSAR-U®, TARABINE PFS®), dasatinib (SPRYCEL®), imatinib mesylate (GLEEVEC®), ponatinib (ICLUSIG®), nilotinib (TASIGNA®) and omacetaxine mepesuccinate (SYNRIBO®).

In some instances, patients may experience allergic reactions to the pharmaceutical compositions of the present invention and/or other anti-cancer agent(s) during or after administration. Therefore, anti-allergic agents may be administered to minimize the risk of an allergic reaction. Suitable anti-allergic agents include corticosteroids, such as dexamethasone (e.g., DECADRON®), beclomethasone (e.g., BECLOVENT®), hydrocortisone (also known as cortisone, hydrocortisone sodium succinate, hydrocortisone sodium phosphate; e.g., ALA-CORT®, hydrocortisone phosphate, Solu-CORTEF®, HYDROCORT Acetate® and LANACORT®), prednisolone (e.g., DELTA-Cortel®, ORAPRED®, PEDIAPRED® and PRELONER), prednisone (e.g., DELTASONE®, LIQUID RED®, METICORTEN® and ORASONE®), methylprednisolone (also known as 6-methylprednisolone, methylprednisolone acetate, methylprednisolone sodium succinate; e.g., DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

In other instances, patients may experience nausea during and after administration of the pharmaceutical composition of the present invention and/or other anti-cancer agent(s). Therefore, anti-emetics may be administered in preventing nausea (upper stomach) and vomiting. Suitable anti-emetics include aprepitant (EMEND®), ondansetron (ZOFRAN®), granisetron HCl (KYTRIL®), lorazepam (ATIVAN®. dexamethasone (DECADRON®), prochlorperazine (COMPAZINE®), casopitant (REZONIC® and Zunrisa®), and combinations thereof.

In yet other instances, medication to alleviate the pain experienced during the treatment period is prescribed to make the patient more comfortable. Common over-the-counter analgesics, such TYLENOL®, are often used. Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) are also useful for moderate or severe pain.

Furthermore, cytoprotective agents (such as neuroprotectants, free-radical scavengers, cardioprotectors, anthracycline extravasation neutralizers, nutrients and the like) may be used as an adjunct therapy to protect normal cells from treatment toxicity and to limit organ toxicities. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In yet another aspect, a pharmaceutical composition of the present invention may be used in combination with known therapeutic processes, for example, with the administration of hormones or in radiation therapy. In certain instances, a pharmaceutical composition of the present invention may be used as a radiosensitizer, especially for the treatment of tumors which exhibit poor sensitivity to radiotherapy.

The doses and dosage regimen of the active ingredients used in the combination therapy may be determined by an attending clinician. In certain embodiments, the pharmaceutical composition described herein (e.g., the First, Second, Third, or Fourth pharmaceutical composition, or other pharmaceutical compositions in Section I) and the additional therapeutic agent(s) are administered in doses commonly employed when such agents are used as monotherapy for treating the disease or condition. In other embodiments, the pharmaceutical composition described herein (e.g., the First, Second, Third, or Fourth pharmaceutical composition, or other pharmaceutical compositions in Section I) and the additional therapeutic agent(s) are administered in doses lower than the doses commonly employed when such agents are used as monotherapy for treating the disease or condition. In certain embodiments, the pharmaceutical composition described herein (e.g., the First, Second, Third, or Fourth pharmaceutical composition, or other pharmaceutical compositions in Section I) and the additional therapeutic agent(s) are present in the same composition, which is suitable for oral administration.

In certain embodiments, the pharmaceutical composition described herein (e.g., the First, Second, Third, or Fourth pharmaceutical composition, or other pharmaceutical compositions in Section I) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of a pharmaceutical composition described herein (e.g., the First, Second, Third, or Fourth pharmaceutical composition, or other pharmaceutical compositions in Section I), and optionally at least one additional therapeutic agent listed above. In certain embodiments, the kit further comprises instructions, such as instructions for treating a disease described herein.

### VII. Administration Aspects

The pharmaceutical compositions of the present invention may be given orally, parenterally, topically, or rectally, and most preferably orally. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage amount of the pharmaceutical compositions of this invention may be varied so as to provide an amount of the active ingredient which is effective to achieve the desired therapeutic response for a particular patient, composition, and mode of administration, without being toxic to the patient.

The selected dosage level will depend upon a variety of factors including the activity of the particular pharmaceutical composition of the present invention employed, the route of administration, the time of administration, the rate of excretion or metabolism of the particular compound being employed, the rate and extent of absorption, the duration of the treatment, other drugs, compounds and/or materials used in combination with the particular pharmaceutical composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the pharmaceutical composition of the invention at levels lower than that required in order to achieve the desired therapeutic effect and gradually increase the dosage until the desired effect is achieved.

In general, a suitable daily dose of a pharmaceutical composition of the invention will be that amount of the pharmaceutical composition which is the lowest dose effective to produce a therapeutic effect. Such an effective dose will generally depend upon the factors described above. Preferably, the pharmaceutical compositions are administered at about 0.01 mg/kg to about 200 mg/kg, more preferably at about 0.1 mg/kg to about 100 mg/kg, even more preferably at about 0.5 mg/kg to about 50 mg/kg; wherein the “mg” refers to the mass of the compound of Formula I, and the “kg” refers to the mass of the patient. When the pharmaceutical compositions described herein are co-administered with another agent (e.g., as sensitizing agents), the effective amount may be less than when the agent is used alone.

If desired, the effective daily dose of the pharmaceutical composition may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a pharmaceutical composition described herein in a therapeutically effective amount for the treatment of a disease or condition described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Preparation of Formulations Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-Tartrate

Four different formulations containing Compound 1 were prepared and tested for stability to storage. Compound 1 is the D-tartaric acid salt of:

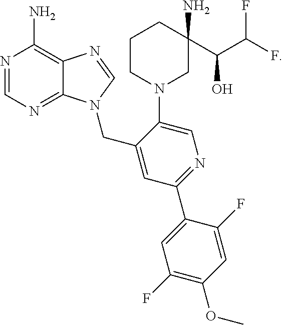 [SMILES: COc1cc(F)c(-c2cc(Cn3cnc4c(N)ncnc43)c(N3CCC[C@](N)([C@H](O)C(F)F)C3)cn2)cc1F]

Experimental procedures and results are provided below.

### Part I—Experimental Procedures

Formulations 1, 2, 3, and 4 set forth below were prepared according to the following general procedure: the required quantity of each component was weighed, screened, and mixed to produce a mixture that was triturated and then sieved through a 30-mesh screen to provide the final formulation.

TABLE 1
Formulations 1 to 4.

| | Amount of the Component (% w/w) | | | |
|---|---|---|---|---|
| | Formulation No. | | | |
| Component in the Formulation | 1 | 2 | 3 | 4 |
| Compound 1 | 2.0 | 2.0 | 2.0 | 2.0 |
| Anhydrous Dibasic Calcium | 71.5 | 72.5 | 72.0 | 0.0 |
| Phosphate | | | | |
| Mannitol | 20.0 | 20.0 | 0.0 | 20.0 |
| Pregelatinized starch | 0.0 | 0.0 | 0.0 | 72.0 |
| Microcrystalline Cellulose | 0.0 | 0.0 | 20.0 | 0.0 |
| Sodium Starch Glycolate | 4.0 | 4.0 | 0.0 | 0.0 |
| Stearic Acid | 1.0 | 0.0 | 0.0 | 0.0 |
| Crospovidone | 0.0 | 0.0 | 4.0 | 4.0 |
| Silicon Dioxide (Syloid244P) | 1.0 | 0.0 | 1.0 | 1.0 |
| Polysorbate 20 | 0.5 | 0.5 | 0.0 | 0.0 |
| Magnesium Stearate | 0.0 | 1.0 | 1.0 | 1.0 |
| Total Mass of the Formulation (g) | 10 | 10 | 10 | 10 |

An aliquot of each of Formulation 1, 2, 3, and 4 was subjected to stability testing. In particular, an amount of each formulation sufficient to provide 11.4 mg of Compound 1 was transferred to glass vials and then the vials were capped. The glass vials were stored at 40° C./75% relative humidity for three to six weeks. Samples were analyzed by HPLC to determine the amount of related substances in the formulation.

### Part II—Results

Results of the stability study are provided in Table 2 below. The results show an increase in the percent of related substances in Formulations 1, 2, and 3 at three weeks. The results show an increase in the percent of related substances in Formulations 1, 2, 3, and 4 at six weeks.

TABLE 2

| | Percentage of Related Substances in the | | |
|---|---|---|---|
| | Formulation (% as Determined by HPLC) | | |
| | At the Beginning of | | |
| Formulation | the Stability Study | 3 Weeks | 6 Weeks |
| No. | (i.e., 0 days storage) | Storage | Storage |
| Compound | 0.42 | 0.42 | 0.41 |
| 1 alone | | | |
| 1 | 0.72 | 1.70 | 1.90 |
| 2 | 0.42 | 0.83 | 1.00 |
| 3 | 0.42 | 0.70 | 1.00 |
| 4 | 0.42 | 0.42 | 0.53 |

### Example 2—Preparation of Additional Formulations Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-Tartrate

Three different formulations containing Compound 1 were prepared and tested for stability to storage. Compound 1 is the D-tartaric acid salt of:

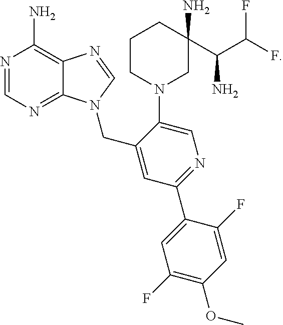 [SMILES: COc1cc(F)c(-c2cc(Cn3cnc4c(N)ncnc43)c(N3CCC[C@](N)([C@H](N)C(F)F)C3)cn2)cc1F]

Experimental procedures and results are provided below.

### Part I—Experimental Procedures

Formulations 5, 6, and 7 set forth below were prepared according to the following general procedure: the required quantity of each component was weighed, screened, and mixed (except for any water) to produce a mixture that was triturated (in the presence of water for Formulation 6 which contains water) and then sieved through a 30-mesh screen to provide the final formulation.

TABLE 1
Formulations 5, 6, and 7.

| | Amount of the Component (% w/w) | | |
|---|---|---|---|
| | Formulation No. | | |
| Component in the Formulation | 5 | 6 | 7 |
| Compound 1 | 1 | 5 | 1 |
| Mannitol | 0 | 0 | 0 |
| Pregelatinized starch | 30 | 30 | 30 |
| Croscarmellose Sodium | 0 | 5 | 5 |
| Hydroxypropyl cellulose | 0 | 5 | 5 |
| Magnesium Stearate | 0 | 0 | 1 |
| Microcrystalline Cellulose | 58 | 49 | 58 |
| Polyvinylpyrrolidone | 5 | 0 | 0 |
| Sodium Starch Glycolate | 5 | 0 | 0 |
| Stearic Acid | 1 | 1 | 0 |
| Purified Water | 0 | 5 | 0 |
| Total Mass of the Formulation (g) | 50 | 10 | 50 |

An aliquot of each of Formulation 5, 6, and 7 was subjected to stability testing. In particular, an amount of each formulation sufficient to provide 11.4 mg of Compound 1 was transferred to a glass vial bottle and then the bottle was capped. The glass vials were stored at (a) 40° C./NMT 25% relative humidity or (b) 40° C./75% relative humidity. Samples were analyzed by HPLC to determine the amount of related substances in the formulation.

### Part II—Results

Results of the stability study are provided in Tables 2 and 3 below. Table 2 presents results for samples stored at 40° C./25% relative humidity. Table 3 presents results for samples stored at 40° C./75% relative humidity.

TABLE 2

| | Percentage of Related Substances in the | | | |
|---|---|---|---|---|
| | Formulation (% as determined by HPLC) | | | |
| | At the Beginning of | | | |
| Formulation | the Stability Study | 2 Weeks | 4 Weeks | 6 Weeks |
| No. | (i.e., 0 days storage) | Storage | Storage | Storage |
| Compound | 0.58 | 0.56 | 0.57 | 0.56 |
| 1 alone | | | | |
| 5 | 0.56 | 0.55 | 0.55 | 0.55 |
| 6 | 0.54 | 0.51 | 0.51 | 0.50 |
| 7 | 0.55 | 0.54 | 0.54 | 0.54 |

TABLE 3

| | Percentage of Related Substances in the | | | |
|---|---|---|---|---|
| | Formulation (% as determined by HPLC) | | | |
| | At the Beginning of | | | |
| Formulation | the Stability Study | 2 Weeks | 4 Weeks | 6 Weeks |
| No. | (i.e., 0 days storage) | Storage | Storage | Storage |
| Compound | 0.58 | 0.57 | 0.56 | 0.57 |
| 1 alone | | | | |
| 5 | 0.56 | 0.55 | 0.56 | 0.62 |
| 6 | 0.54 | 0.51 | 0.51 | 0.51 |
| 7 | 0.55 | 0.54 | 0.55 | 0.61 |

### Example 3—Preparation of Formulations Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol Fumarate

Three different formulations containing Compound 2 were prepared and tested for stability to storage. Compound 2 is the fumaric acid salt of:

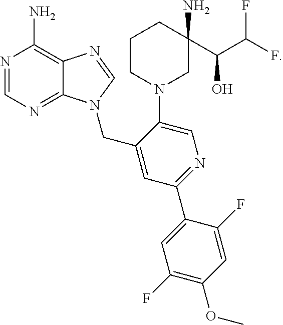 [SMILES: COc1cc(F)c(-c2cc(Cn3cnc4c(N)ncnc43)c(N3CCC[C@](N)([C@H](O)C(F)F)C3)cn2)cc1F]

Experimental procedures and results are provided below.

### Part I—Experimental Procedures

Formulations 8, 9, and 10 set forth below were prepared according to the following general procedure: the required quantity of each component was weighed, screened, and mixed (except for any water) to produce a mixture that was triturated (in the presence of water for Formulation 9 which contains water) and then sieved through a 30-mesh screen to provide the final formulation.

TABLE 1
Formulations 8, 9, and 10.

| | Amount of the Component (% w/w) | | |
|---|---|---|---|
| | Formulation No. | | |
| Component in the Formulation | 8 | 9 | 10 |
| Compound 2 | 1.0 | 5.0 | 1.0 |
| Mannitol | 0.0 | 0.0 | 0.0 |
| Pregelatinized starch | 30.0 | 30.0 | 30.0 |
| Croscarmellose Sodium | 0.0 | 5.0 | 5.0 |
| Hydroxypropyl cellulose | 0.0 | 5.0 | 5.0 |
| Magnesium Stearate | 0.0 | 0.0 | 1.0 |
| Microcrystalline Cellulose | 58.0 | 49.0 | 58.0 |
| Polyvinylpyrrolidone | 5.0 | 0.0 | 0.0 |
| Sodium Starch Glycolate | 5.0 | 0.0 | 0.0 |
| Stearic Acid | 1.0 | 1.0 | 0.0 |
| Purified Water | 0.0 | 5.0 | 0.0 |
| Total Mass of the Formulation (g) | 50 | 10 | 50 |

An aliquot of each of Formulation 8, 9, and 10 was subjected to stability testing. In particular, an amount of each formulation sufficient to provide 14.3 mg of Compound 2 was transferred to a glass vial and then the vial was capped. The glass vials were stored at (a) 40° C./NMT 25% relative humidity or (b) 40° C./75% relative humidity. Samples were analyzed to determine the amount of related substances in the formulation.

### Part II—Results

Results of the stability study are provided in Tables 2 and 3 below. Table 2 presents results for samples stored at 40° C./25% relative humidity. Table 3 presents results for samples stored at 40° C./75% relative humidity.

TABLE 2

| | Percentage of Related Substances in the | | | |
|---|---|---|---|---|
| | Formulation (% as determined by HPLC) | | | |
| | At the Beginning of | | | |
| Formulation | the Stability Study | 2 Weeks | 4 Weeks | 6 Weeks |
| No. | (i.e., 0 days storage) | Storage | Storage | Storage |
| Compound | 0.56 | 0.55 | 0.55 | 0.56 |
| 2 alone | | | | |
| 8 | 0.54 | 0.54 | 0.55 | 0.54 |
| 9 | 0.53 | 0.50 | 0.50 | 0.49 |
| 10 | 0.53 | 0.53 | 0.53 | 0.53 |

TABLE 3

| | Percentage of Related Substances in the | | | |
|---|---|---|---|---|
| | Formulation (% as determined by HPLC) | | | |
| | At the Beginning of | | | |
| Formulation | the Stability Study | 2 Weeks | 4 Weeks | 6 Weeks |
| No. | (i.e., 0 days storage) | Storage | Storage | Storage |
| Compound | 0.56 | 0.55 | 0.56 | 0.55 |
| 2 alone | | | | |
| 8 | 0.54 | 0.54 | 0.55 | 0.58 |
| 9 | 0.53 | 0.50 | 0.51 | 0.46 |
| 10 | 0.53 | 0.53 | 0.59 | 0.61 |

### Example 4—Preparation of Tablets of a Formulation Containing (S)-1-((R)-3-amino-1-(4-((6-amino-9H-purin-9-yl)methyl)-6-(2,5-difluoro-4-methoxyphenyl) pyridin-3-yl)piperidin-3-yl)-2,2-difluoroethan-1-ol D-Tartrate

Two different formulations containing Compound 1 were prepared and compressed to form tablets. Compound 1 is the D-tartaric acid salt of:

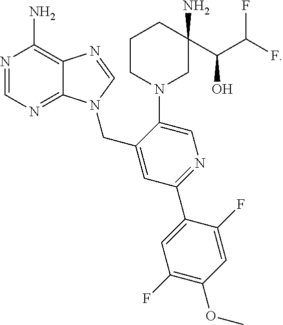 [SMILES: COc1cc(F)c(-c2cc(Cn3cnc4c(N)ncnc43)c(N3CCC[C@](N)([C@H](O)C(F)F)C3)cn2)cc1F]

Experimental procedures and results are provided below.

### Part I—Experimental Procedures

Formulations 11 and 12 set forth below were prepared according to the following general procedure: the required quantity of each component was weighed, screened, and mixed to produce a mixture that was triturated and then sieved through a 30-mesh screen to provide the final formulation.

TABLE 1
Formulations 11 and 12.

| | Amount of the Component (% w/w) | |
|---|---|---|
| | Formulation No. | |
| Component in the Formulation | 11 | 12 |
| Compound 1 | 20 | 20 |
| Mannitol | 25 | 30 |
| Anhydrous dibasic calcium | 25 | 18 |
| phosphate | | |
| Pregelatinized starch | 18 | 20 |
| Croscarmellose sodium | 4.5 | 4.5 |
| Crospovidone | 5.0 | 5.0 |
| Silicon dioxide | 1.5 | 1.5 |
| Magnesium stearate | 1.0 | 1.0 |
| Total Mass of the Formulation | 500 | 500 |
| (mg) | | |

Tablets were formed from Formulation 11. Additionally, tablets were formed from Formulation 12. Circular, flat tablets were prepared by compressing the formulations using a single-punch manual press.

### Part II—Results

To generate a tablet from Formulation 11 required applying a pressure of at least 5,100 psi. Tablets formed from Formulation 11 using a pressure of 5,100 psi had the properties set forth in Table 2 below. Tablets formed from Formulation 11 using a pressure of 8,000 psi had the properties set forth in Table 2 below.

TABLE 2

Data
Compression Pressure Used
to Form the Tablet (PSI)

| Characteristic | 5,100 | 8,000 |
|---|---|---|
| Average weight (mg) | 501.5 | 502.6 |
| Average hardness (kP) | 13.4 | 17.6 |
| Average thickness (mm) | 4.20 | 4.10 |
| Average disintegration time | 0:01:09 | 0:01:28 |
| (h:min:sec) | | |

To generate a tablet from Formulation 12 required applying a pressure of at least 5,300 psi. Tablets formed from Formulation 12 using a pressure of 5,300 psi had the properties set forth in Table 3 below. Tablets formed from Formulation 12 using a pressure of 8,000 psi had the properties set forth in Table 3 below.

TABLE 3

Data
Compression Pressure Used
to Form the Tablet (PSI)

| Characteristic | 5,300 | 8,000 |
|---|---|---|
| Average Weight (mg) | 499.4 | 499.7 |
| Average hardness (kP) | 15.2 | 15.8 |
| Average thickness (mm) | 4.11 | 4.02 |
| Average disintegration time | 0:01:11 | 0:01:37 |
| (h:min:sec) | | |

### Example 5—Preparation of Tablets of a Different Formulation Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-Tartrate

A formulation containing Compound 1 was prepared and compressed to form tablets. Compound 1 is the D-tartaric acid salt of:

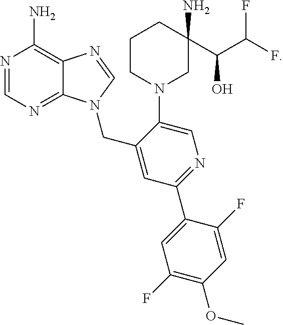 [SMILES: COc1cc(F)c(-c2cc(Cn3cnc4c(N)ncnc43)c(N3CCC[C@](N)([C@H](O)C(F)F)C3)cn2)cc1F]

Experimental procedures and results are provided below.

### Part I—Experimental Procedures

Formulation 13 set forth below was prepared according to the following general procedure: the required quantity of each component was weighed, screened, and mixed to produce a mixture that was triturated and then sieved through a 30-mesh screen to provide the final formulation.

TABLE 1
Formulation 13.

| | Amount of the |
|---|---|
| Component in the Formulation | Component (% w/w) |
| Compound 1 | 23.2 |
| Microcrystalline Cellulose | 51.8 |
| Pregelatinized starch | 20.0 |
| Croscarmellose sodium | 4.00 |
| Magnesium stearate | 1.00 |
| Total Mass of the Formulation (mg) | 500 |

Tablets were formed from Formulation 13. Tablets were prepared by compressing the formulation using a single-punch manual press.

### Part II—Results

Tablets from Formulation 13 could be prepared by applying a pressure of ≤3,200 psi. Tablets formed from Formulation 13 using a pressure of 2,200 psi had the properties set forth in Table 3 below. Circular, flat tablets formed from Formulation 13 using a pressure of 3,200 psi had the properties set forth in Table 3 below.

TABLE 3

Data
Compression Pressure Used
to Form the Tablet (PSI)

| Characteristic | 2,200 | 3,200 |
|---|---|---|
| Average weight (mg) | 501.0 | 501.3 |
| Average hardness (kP) | 14.0 | 20.5 |
| Average thickness (mm) | 4.51 | 4.29 |
| Average disintegration time | 0:00:40 | 0:00:52 |
| (h:min:sec) | | |

### Example 6—Preparation of Tablets of a Formulation Containing (S)-1-((R)-3-amino-1-(4-((6-amino-9H-purin-9-yl)methyl)-6-(2,5-difluoro-4-methoxyphenyl) pyridin-3-yl)piperidin-3-yl)-2,2-difluoroethan-1-ol Fumarate

Two different formulations containing Compound 2 were prepared and compressed to form tablets. Compound 2 is the fumaric acid salt of:

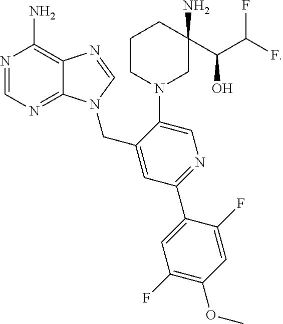 [SMILES: COc1cc(F)c(-c2cc(Cn3cnc4c(N)ncnc43)c(N3CCC[C@](N)([C@H](O)C(F)F)C3)cn2)cc1F]

Experimental procedures and results are provided below.

### Part I—Experimental Procedures

Formulations 14 and 15 set forth below were prepared according to the following general procedure: the required quantity of each component was weighed, screened, and mixed to produce a mixture that was triturated and then sieved through a 30-mesh screen to provide the final formulation.

TABLE 1
Formulation 14.

| | Amount of the |
|---|---|
| Component in the Formulation | Component (% w/w) |
| Compound 2 | 20.0 |
| Microcrystalline Cellulose | 50.0 |
| Pregelatinized starch | 24.0 |
| Croscarmellose sodium | 5.0 |
| Magnesium stearate | 1.0 |
| Total Mass of the Formulation (mg) | 500 |

TABLE 2
Formulation 15.

| | Amount of the |
|---|---|
| Component in the Formulation | Component (% w/w) |
| Compound 2 | 26.6 |
| Microcrystalline Cellulose | 43.4 |
| Pregelatinized starch | 24.0 |
| Croscarmellose sodium | 5.0 |
| Magnesium stearate | 1.0 |
| Total Mass of the Formulation (mg) | 500 |

Tablets were formed from Formulation 14. Additionally, tablets were formed from Formulation 15. Circular, flat tablets were prepared by compressing the formulations using a single-punch manual press.

### Part II—Results

Tablets from Formulation 14 and Formulation 15 could be prepared by applying a pressure of ≤3,400 psi. Tablets formed from Formulation 14 had the properties set forth in Table 3 below. Tablets formed from Formulation 15 had the properties set forth in Table 4 below.

TABLE 3

Data
Compression Pressure Used
to Prepare the Tablet (PSI)

| Characteristic | 2,400 | 3,200 |
|---|---|---|
| Average weight, mg (n = 10) | 498.1 | 501.0 |
| Average hardness, kP (n = 10) | 4.50 | 4.33 |
| Average thickness, mm (n = 10) | 11.8 | 16.7 |
| Disintegration time h:min:sec | 0:00:59 | 0:00:50 |
| (n = 6 without discs) | | |

TABLE 4

Data
Compression Pressure Used
to Prepare the Tablet (PSI)

| Characteristic | 2,200 | 2,600 | 3,400 |
|---|---|---|---|
| Average weight, mg (n = 10) | 510.3 | 510.0 | 505.8 |
| Average hardness, kP (n = 10) | 4.54 | 4.33 | 4.33 |
| Average thickness, mm (n = 10) | 12.3 | 15.6 | 19.5 |
| Disintegration time h:min:sec | 0:1:00 | 0:1:05 | 0:1:10 |
| (n = 6 without discs) | | | |

### Example 7—Tablet Preparation Using a Rotary Tablet Machine for a Formulation Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-Tartrate

A formulation containing Compound 1 were prepared, and tablets were made using a rotary tablet machine. Compound 1 is the D-tartaric acid salt of:

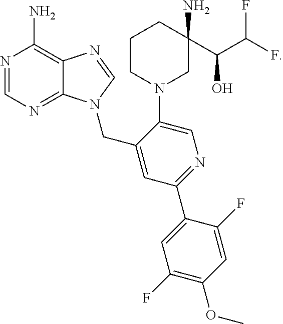 [SMILES: COc1cc(F)c(-c2cc(Cn3cnc4c(N)ncnc43)c(N3CCC[C@](N)([C@H](O)C(F)F)C3)cn2)cc1F]

Experimental procedures and results are provided below.

Formulation 16 set forth below was prepared according to the following general procedure: Compound 1 and the excipients were blended in a diffusion mixer (V-Blender) to produce a mixture, and then the mixture was screened using a co-mil with a 991-micron screen at 1200 rpm, to thereby provide Formulation 16. The components in Formulation 16 are set forth in Table 1 below.

TABLE 1
Formulation 16.

| Component in the Formulation | Percent w/w (%) |
|---|---|
| Compound 1 | 21.8 |
| Microcrystalline Cellulose | 53.2 |
| Pregelatinized starch | 20.0 |
| Croscarmellose sodium | 4.0 |
| Magnesium stearate | 1.0 |

Formulation 16 was fed to a rotary tablet machine for the purpose of making tablets. The target weight for the tablets was 150 mg, and the target hardness for the tablets was 8 kP. Formulation 16 was visually observed to have poor flow properties, which adversely impacted ability to manufacture tablets on a rotary tablet machine. The poor flow properties of Formulation 16 contributed to substantial variation in the process and specifications of the resulting tablets. For example, for the tablets prepared, the weight of the tablets varied from 50 mg to 90 mg. As such, the target weight of the tablets could not be achieved.

### Example 8—Tablet Preparation Using a Rotary Tablet Machine for a Formulation Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol Fumarate

A formulation containing Compound 2 was prepared, and tablets were made using a rotary tablet machine. Compound 2 is the fumaric acid salt of:

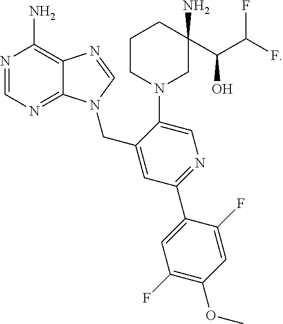 [SMILES: COc1cc(F)c(-c2cc(Cn3cnc4c(N)ncnc43)c(N3CCC[C@](N)([C@H](O)C(F)F)C3)cn2)cc1F]

Experimental procedures and results are provided below.

Formulation 17 set forth below was prepared according to the following general procedure: Compound 2 and the excipients were blended in a diffusion mixer (V-Blender) to produce a mixture, and then the mixture was screened using a co-mil with a 991-micron screen at 1200 rpm, to thereby provide Formulation 17. The components in Formulation 17 are set forth in Table 1 below.

TABLE 1
Formulation 17.

| Component in the Formulation | Percent w/w (%) |
|---|---|
| Compound 2 | 23.2 |
| Microcrystalline Cellulose | 51.8 |
| Pregelatinized starch | 20.0 |
| Croscarmellose sodium | 4.0 |
| Magnesium stearate | 1.0 |

Formulation 17 was fed to a rotary tablet machine for the purpose of making tablets. The target weight for each tablet was 900 mg, and the target hardness for each tablet was 18 kP. Formulation 17 was visually observed to have poor flow properties, which adversely impacted ability to make tablets using the rotary tablet machine. The poor flow properties of Formulation 17 contributed to substantial variation in the process and specifications of the resulting tablets. The weight variation was observed to be more than the specified range (900±45 mg) and target hardness for the tablets was not achieved.

### Example 9—Tablet Preparation Using Dry Granulation Procedure for a Formulation Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol D-Tartrate

Formulation 16 was subjected to roller compactor (gap width: 2 mm; compaction force: 5 kN/cm; roller speed: 1.5 rpm, screen size: 1.25 mm) to produce a granulate. The granulate was mixed with magnesium stearate (at a ratio of 99.5 to 0.5 by weight granulate to magnesium stearate) to produce a final mixture. The final mixture was visually observed to have satisfactory flow properties, and tablets were made from the final mixture using a rotary tablet machine without difficulty.

Table 1 below shows particle sizes in the formulation before and after roller compaction. The data in Table 1 show that the size of particles in the formulation was substantially larger after subjecting the formulation to roller compaction. In particular, the average particle size in the formulation after roller compaction (d₅₀ was approximately 300 microns) was approximately four-fold larger than the average particle size in the formulation prior to roller compaction (d₅₀ was approximately 75 microns). An aliquot of Formulation 16 was compressed to a target weight of 150 mg to form a tablet, and the properties of the tablet are set forth in Table 2.

TABLE 1

| | Formulation Before | Formulation After |
|---|---|---|
| | Roller Compaction | Roller Compaction |
| Mesh | Cumulative % of | Cumulative % of |
| Size | Material Retained | Material Retained |
| 20 | 1.2 | 25.3 |
| 40 | 4.7 | 53.7 |
| 60 | 10.1 | 62.7 |
| 80 | 15.0 | 68.2 |
| 100 | 20.7 | 72.6 |
| 200 | 39.2 | 86.7 |
| 325 | 100 | 100.0 |

TABLE 2

| Characteristic | Data | |
|---|---|---|
| Average weight, mg (range, n = 10) | 151.0 | (147.6-153.6) |
| Average hardness, kP (range, n = 10) | 8.7 | (7.6-9.7) |
| Average thickness, mm (range, n = 10) | 3.54 | (3.49-3.60) |

| | |
|---|---|
| Disintegration time h:min:sec | 0:02:11 |
| (n = 6 without discs) | |

### Example 10—Tablet Preparation Using Dry Granulation Procedure for a Formulation Containing (S)-1-((R)-3-amino-1-(4-((6-amino-9H-purin-9-yl)methyl)-6-(2,5-difluoro-4-methoxyphenyl)pyridin-3-yl)piperidin-3-yl)-2,2-difluoroethan-1-ol Fumarate

Formulation 17 was subjected to roller compactor (gap width: 2 mm; compaction force: 5 kN/cm; roller speed: 1.5 rpm, screen size: 1.25 mm) to produce a granulate. The granulate was mixed with magnesium stearate (at a ratio of 99.5 to 0.5 by weight of granulate to magnesium stearate) to produce a final mixture. The final mixture was visually observed to have satisfactory flow properties, and tablets were made from the final mixture using a rotary tablet machine without difficulty. Aliquots of Formulation 17 were compressed to a target weight of 150 mg and 900 mg to provide tablets. Properties of the tablets are set forth in Table 1.

TABLE 1

| | Data for Tablets | Data for Tablets |
|---|---|---|
| | Having a Target | Having a Target |
| Characteristic | Weight of 150 mg | Weight of 900 mg |
| Average weight, mg | 151.1 | 903.0 |
| (range, n = 10) | (146.5-153.8) | (895.0-914.0) |
| Average hardness, kP | 7.7 | 18.9 |
| (range, n = 10) | (7.0-8.5) | (15.5-23.0) |
| Average thickness, mm | 3.53 | 6.67 |
| (range, n = 10) | (3.44-3.61) | (6.50-6.81) |
| Disintegration time, h:min:sec | 0:01:50 | 0:00:51 |
| (n = 6 without discs) | | |

### Example 11—Preparation of Film-Coated Tablets

Tablets containing Compound 1 prepared according to the procedure in Example 9 above were coated with a film. Procedures and results are provided below. Compound 1 is the D-tartaric acid salt of:

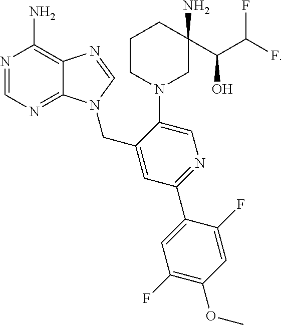 [SMILES: COc1cc(F)c(-c2cc(Cn3cnc4c(N)ncnc43)c(N3CCC[C@](N)([C@H](O)C(F)F)C3)cn2)cc1F]

Tablets having a weight of 900 mg were spray-coated to provide an Opadry Pink film coating on the tablet. Procedurally, tablets were heated and then sprayed with a spray-coating mixture (where the mixture contained water, hypromellose, lactose monohydrate, titanium dioxide, polyethylene glycol, red iron oxide, triacetin, and a colorant) to achieve a weight gain due to the spray coating of about 4% w/w, and the resulting film-coated tablets were allowed to dry. The resulting film-coated tablets had a film coating that was 4% w/w of the overall tablet.

### Example 12—Tablet Stability Study

Tablets containing Compound 1 prepared based on procedures described in Examples 9 and 11 above were subjected to a stability study. Tablets were stored at a temperature of 25° C. at 60% relative humidity for up to 18 months. The physical appearance, purity, and water content of tablets was analyzed at the intervals set forth in Table 1 below. Tablets used in the study were (i) tablets containing 25 mg of Compound 1, (ii) tablets containing 150 mg of Compound 1, or (iii) tablets containing 150 mg of Compound 1 where the tablet had a film coating (which corresponded to the film-coated tablets described in Example 11).

Compound 1 is the D-tartaric acid salt of:

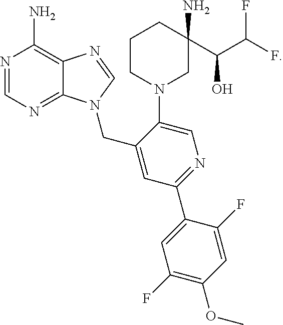 [SMILES: COc1cc(F)c(-c2cc(Cn3cnc4c(N)ncnc43)c(N3CCC[C@](N)([C@H](O)C(F)F)C3)cn2)cc1F]

TABLE 1

| | Stability Study Results | | |
|---|---|---|---|
| | | Amount of Compound 1 | |
| | | Relative to Reference | Water Content, |
| Time | Appearance | Standard (Percent) | Percent (w/w) |

Tablet Containing 25 mg of Compound 1

| | | | |
|---|---|---|---|
| T = 0 | Round white | N/A | 3.3 |
| | tablets | | |
| T = 3 | Round white | 98.3 | 4.4 |
| months | tablets | | |
| T = 6 | Round white | 99.6 | 3.8 |
| months | tablets | | |
| T = 9 | Round white | 99.9 | 4.0 |
| months | tablets | | |
| T = 12 | Round white | 100.1 | 4.0 |
| months | tablets | | |
| T = 18 | Round white | 100.9 | 4.4 |
| months | tablets | | |

Tablet Containing 150 mg of Compound 1

| | | | |
|---|---|---|---|
| T = 0 | Capsule shaped | N/A | 3.4 |
| | white tablets | | |
| T = 3 | Capsule shaped | 98.5 | 3.7 |
| months | white tablets | | |
| T = 6 | Capsule shaped | 99.2 | 3.5 |
| months | white tablets | | |
| T = 9 | Capsule shaped | 99.6 | 3.4 |
| months | white tablets | | |
| T = 12 | Capsule shaped | 98.6 | 3.1 |
| months | white tablets | | |
| T = 18 | Capsule shaped | 100.1 | 3.8 |
| months | white tablets | | |

Film-Coated Tablet Containing 150 mg of Compound 1

| | | | |
|---|---|---|---|
| T = 0 | Capsule shaped, | 102.6 | 3.5 |
| | light pink tablets | | |
| T = 3 | Capsule shaped, | 101.4 | 4.1 |
| months | light pink tablets | | |
| T = 6 | Capsule shaped, | 102.9 | 3.8 |
| months | light pink tablets | | |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A pharmaceutical composition, comprising:
(a) at least 10% w/w of a compound of Formula I, wherein Formula I is represented by: 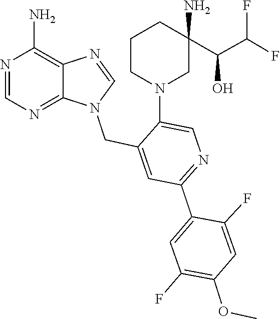 [SMILES: COc1cc(F)c(-c2cc(Cn3cnc4c(N)ncnc43)c(N3CCC[C@](N)([C@H](O)C(F)F)C3)cn2)cc1F]
or a pharmaceutically acceptable salt thereof;
(b) at least 40% w/w microcrystalline cellulose;
(c) at least 10% w/w pregelatinized starch;
(d) at least 2% w/w croscarmellose or a pharmaceutically acceptable salt thereof; and
(e) at least 0.75% w/w stearic acid or a pharmaceutically acceptable salt thereof.

2. (canceled)

3. The pharmaceutical composition of claim 1, wherein the pharmaceutical composition comprises from 45% to 65% w/w microcrystalline cellulose.

4. The pharmaceutical composition of claim 1, wherein the pharmaceutical composition comprises from 50% to 56% w/w microcrystalline cellulose.

5. (canceled)

6. (canceled)

7. (canceled)

8. The pharmaceutical composition of claim 1, wherein the pharmaceutical composition comprises at least 20% w/w pregelatinized starch.

9. The pharmaceutical composition of claim 1, wherein the pharmaceutical composition comprises from 15% to 25% w/w pregelatinized starch.

10. The pharmaceutical composition of claim 1, wherein the pharmaceutical composition comprises from 18% to 22% w/w pregelatinized starch.

11. (canceled)

12. (canceled)

13. The pharmaceutical composition of claim 1, wherein the pharmaceutical composition comprises from 3% w/w to 5% w/w croscarmellose or a pharmaceutically acceptable salt thereof.

14. (canceled)

15. The pharmaceutical composition of claim 1, wherein the croscarmellose or a pharmaceutically acceptable salt thereof is croscarmellose sodium.

16. (canceled)

17. (canceled)

18. The pharmaceutical composition of claim 1, wherein the pharmaceutical composition comprises from 1% to 2% w/w of stearic acid or a pharmaceutically acceptable salt thereof.

19. (canceled)

20. The pharmaceutical composition of claim 1, wherein the stearic acid or a pharmaceutically acceptable salt thereof is an alkaline earth metal salt of stearic acid.

21. The pharmaceutical composition of claim 1, wherein the stearic acid or a pharmaceutically acceptable salt thereof is magnesium stearate.

22. (canceled)

23. The pharmaceutical composition of claim 1, wherein the pharmaceutical composition comprises at least 20% w/w of a compound of Formula I.

24. (canceled)

25. The pharmaceutical composition of claim 1, wherein the pharmaceutical composition comprises from 18% to 24% w/w of a compound of Formula I.

26. The pharmaceutical composition of claim 1, wherein the pharmaceutical composition comprises from 20% to 22% w/w of a compound of Formula I.

27. (canceled)

28. The pharmaceutical composition of claim 1, wherein the compound of Formula I is a tartaric acid salt of

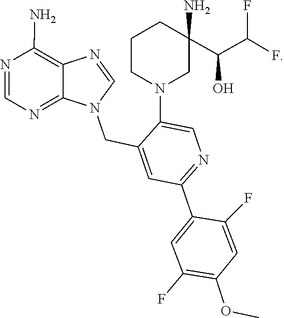 [SMILES: COc1cc(F)c(-c2cc(Cn3cnc4c(N)ncnc43)c(N3CCC[C@](N)([C@H](O)C(F)F)C3)cn2)cc1F]

29. The pharmaceutical composition of claim 1, wherein the compound of Formula I is a D-tartaric acid salt of

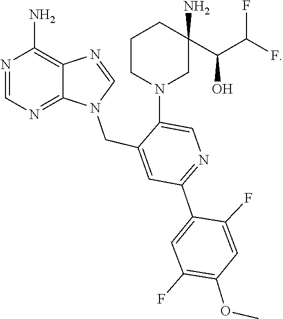 [SMILES: COc1cc(F)c(-c2cc(Cn3cnc4c(N)ncnc43)c(N3CCC[C@](N)([C@H](O)C(F)F)C3)cn2)cc1F]

30. (canceled)

31. (canceled)

32. A pharmaceutical composition, comprising:
(a) from 17% w/w to 25% w/w of a compound of Formula I, wherein Formula I is a D-tartaric acid salt of 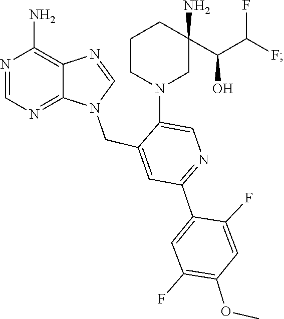 [SMILES: COc1cc(F)c(-c2cc(Cn3cnc4c(N)ncnc43)c(N3CCC[C@](N)([C@H](O)C(F)F)C3)cn2)cc1F]
(b) from 51% to 56% w/w microcrystalline cellulose;
(c) about 20% w/w pregelatinized starch;
(d) about 4% w/w croscarmellose sodium; and
(e) about 1.5% w/w magnesium stearate.

33. (canceled)

34. The pharmaceutical composition of claim 32, wherein the pharmaceutical composition comprises from 20% w/w to 22% w/w of the compound of Formula I.

35. The pharmaceutical composition of claim 32, wherein the pharmaceutical composition comprises about 21% w/w of the compound of Formula I.

36. (canceled)

37. The pharmaceutical composition of claim 34, wherein the pharmaceutical composition comprises from 53% to 54% w/w microcrystalline cellulose.

38. The pharmaceutical composition of claim 35, wherein the pharmaceutical composition comprises 53% w/w microcrystalline cellulose.

39. A pharmaceutical composition, comprising:
(a) at least 10% w/w of a compound of Formula I, wherein Formula I is represented by: 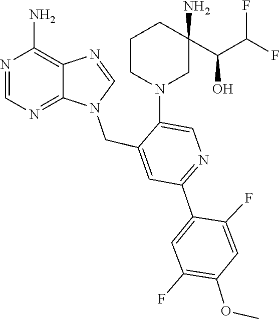 [SMILES: COc1cc(F)c(-c2cc(Cn3cnc4c(N)ncnc43)c(N3CCC[C@](N)([C@H](O)C(F)F)C3)cn2)cc1F]
or a pharmaceutically acceptable salt thereof; and
(b) at least 60% w/w of a diluent selected from microcrystalline cellulose, pregelatinized starch, or a combination thereof.

40-51. (canceled)

52. A tablet for oral administration, comprising a pharmaceutical composition of claim 1.

53. (canceled)

54. A method of preparing a pharmaceutical composition, comprising the steps of:
(i) providing a first mixture comprising:
(a) at least 10% w/w of a compound of Formula I, wherein Formula I is represented by: 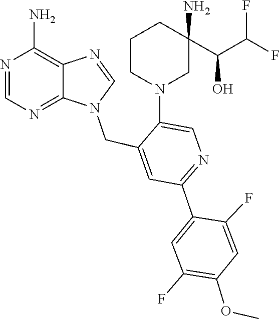 [SMILES: COc1cc(F)c(-c2cc(Cn3cnc4c(N)ncnc43)c(N3CCC[C@](N)([C@H](O)C(F)F)C3)cn2)cc1F]
or a pharmaceutically acceptable salt thereof;
(b) at least 40% w/w microcrystalline cellulose;
(c) at least 10% w/w pregelatinized starch;
(d) at least 2% w/w croscarmellose or a pharmaceutically acceptable salt thereof; and
(e) at least 0.75% w/w stearic acid or a pharmaceutically acceptable salt thereof;
(ii) subjecting the first mixture to roller compaction to produce a compacted mixture; and
(iii) subjecting the compacted mixture to compressive force to produce a compressed pharmaceutical composition.

55. (canceled)

56. (canceled)

57. A method for treating a disease or condition mediated by nuclear SET domain-containing protein 2 (NSD2), comprising administering to a subject in need thereof a therapeutically effective amount of a pharmaceutical composition of claim 1 to treat the disease or condition.

58. (canceled)

59. The method of claim 57, wherein said disease or condition mediated by NSD2 is selected from a solid tumor, leukemia, myeloma, lymphoma, and hypertension.

60. The method of claim 57, wherein said disease or condition mediated by NSD2 is myeloma.

61. The method of claim 57, wherein said disease or condition mediated by NSD2 is breast cancer, cervical cancer, skin cancer, ovarian cancer, gastric cancer, prostate cancer, pancreatic cancer, lung cancer, hepatocellular carcinoma, head and neck cancer, peripheral nerve sheath tumor, osteosarcoma, multiple myeloma, neuroblastoma, leukemia, non-Hodgkin's lymphoma, or pulmonary arterial hypertension.

62. (canceled)

63. (canceled)

64. A method of inhibiting the activity of nuclear SET domain-containing protein 2 (NSD2), comprising contacting a NSD2 with an effective amount of a pharmaceutical composition of claim 1 to inhibit the activity of said NSD2.
